(19) United States
(12) Patent Application Publication
(10) Pub. No.: US 2011/0190529 A1
(43) Pub. Date: Aug. 4, 2011

(54) METHOD FOR PREPARING THE ETHYL ESTER OF 4-[TRANS-4-[(PHENYLMETHYL)-AMINO]CYCLOHEXYL]BENZOIC ACID AND THE HEMIFUMARATE SALT THEREOF

(75) Inventor: Jose Ruiz-Montes, Paris (FR)

(73) Assignee: SANOFI-AVENTIS, Paris (FR)

(21) Appl. No.: 13/059,871

(22) PCT Filed: Aug. 18, 2009
(86) PCT No.: PCT/FR2009/001013

§ 371 Date: Apr. 14, 2011

(30) Foreign Application Priority Data
Aug. 21, 2008 (FR) 0804665

(51) Int. Cl. C07C 303/36; C07C 227/22; C07C 227/16; C07C 229/46; C07C 303/40

(52) U.S. Cl. 560/13; 560/48

(57) ABSTRACT

The present invention relates to the compound of formula (I) and to the use thereof as an intermediate for the synthesis of the compound of formula (A) or pharmaceutically acceptable salts thereof: formulae (II).

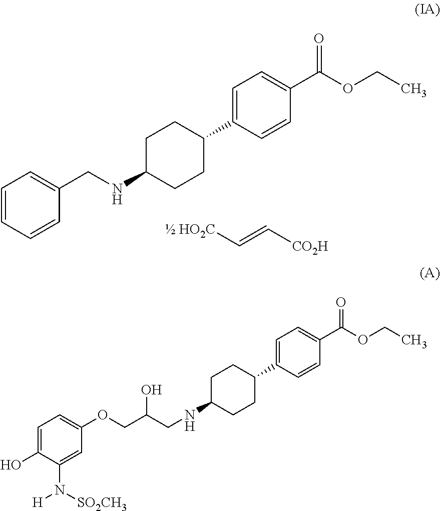

The subject of the present invention is a method for preparing the compound of formula (I):

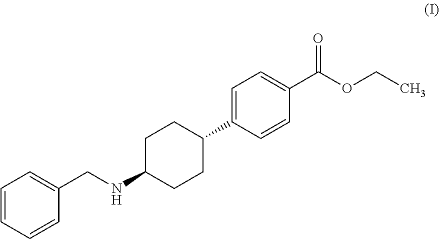

The subject of the present invention is also the hemifumarate of the compound of formula (I), corresponding to formula (IA):

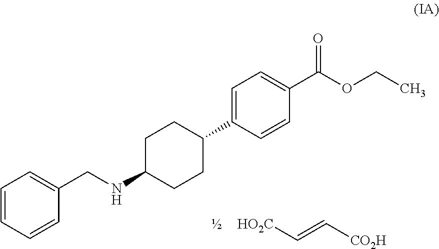

The compound of formula (I) is described in patent applications WO 03/099772, WO 03/099819 and WO 02/44139, where it can be used as intermediate for preparing the compound of formula (A) or pharmaceutically acceptable salts thereof:

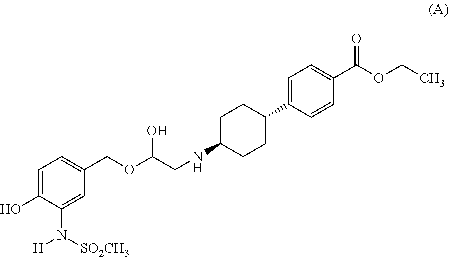

According to the method of synthesis described in the abovementioned patent applications, the compound of formula (I) is synthesized by reacting a compound of formula (II):

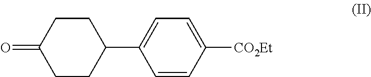

with a compound of formula (III):

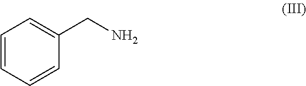

A cis/trans mixture is obtained and the compound of formula (I) (trans isomer) is obtained and separated from the cis isomer by silica chromatography.

It is therefore desirable to provide a novel improved method which can be transposed to the industrial scale and which makes it possible to be free of the silica chromatography step.

It has now been discovered, and this is one of the subjects of the present invention, that salification of the reaction crude resulting from the reaction of the compound (II) with the compound (III), by means of fumaric acid, makes it possible to obtain the crystalline compound of formula (I), in trans configuration, with good chemical purity. This method thus makes it possible to easily separate the compound of formula (I), without having to resort to the silica chromatography step of the prior art.

According to the present invention, a compound of formula (I) is prepared via the formation of the compound of general formula (IA):

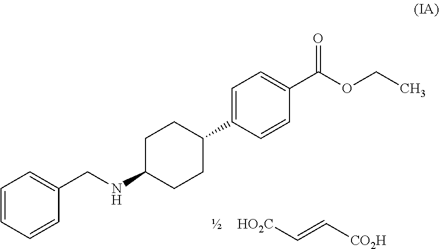

According to the invention, the method for preparing the compound of formula (IA) comprises the reaction of a compound of formula (II) with a compound of formula (III)

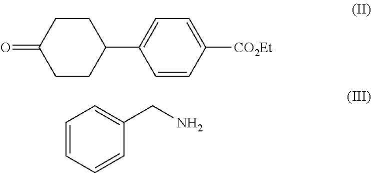

characterized in that:
said reaction is followed by a step of salification by addition of fumaric acid.

At the end of the reaction of a compound of formula (II) with the compound of formula (III), the compound of formula (I) is obtained in racemic form (mixture of the cis/trans isomers). The addition of fumaric acid makes it possible to separate the hemifumarate addition salt of the trans form, of formula (IA), since said salt precipitates and can therefore be easily separated from the cis isomer.

Generally, half an equivalent of fumaric acid, relative to the compound of formula (II), is added.

According to the invention, the method for preparing the compound of formula (I) comprises the step of addition of a base to the compound of formula (IA).

During the addition of a base, the compound of formula (IA) is converted to its basic form, i.e. the compound of formula (I).

The reaction of the compound of formula (II) with the compound of formula (III) can be carried out as described in WO 02/44139. This reaction can be carried out in an organic solvent, at a temperature between ambient temperature and the reflux temperature of the solvent. The term “ambient temperature” is intended to mean a temperature between 18° C. and 25° C. By way of solvent, mention may in particular be made of ethanol or trimethyl orthoformate. Advantageously, sodium borohydride (NaBH₄) or a derivative thereof (such as NaBH₃CN) is added. The reaction mixture is preferably left stirring at a temperature between 0° C. and ambient temperature. The reaction mixture obtained can be used as is for the rest of the reaction or can be extracted by means of an organic solvent, such as ethyl acetate, washed, dried and evaporated according to routine methods, in order to be used in the subsequent salification step.

Advantageously, the salification step can be carried out in an organic solvent, at a temperature between ambient temperature and the reflux temperature of the solvent. By way of solvent, mention may in particular be made of ethanol.

After addition of the fumaric acid, the solubility of the reaction medium under hot conditions, and the formation of a precipitate during cooling, are generally observed. The crystals obtained correspond to the hemifumarate salt of the trans compound of formula (I) (compound (IA)).

The compound of formula (I) can be obtained according to the methods known to those skilled in the art, for example by addition of a base, in an organic solvent.

By way of base, mention may in particular be made of organic bases, such as a solution of aqueous ammonia or NH₄OH. As organic solvent, mention may in particular be made of (C₁-C₄) alkyl acetates, esters, aromatic solvents such as toluene, ethers such as isopropyl ether or tert-butyl methyl ether (TBME), dichloromethane, methyltetrahydrofuran (THF) and also methoxycyclopropane.

The compound of formula (I) thus obtained can be used in the subsequent steps of the preparation method resulting in the product of formula (A) according to the methods described in WO 02/44139.

The method for preparing the compound of formula (I) can be carried out directly or can comprise the step of separating the compound of formula (IA) at the end of the salification and before the addition of base. This separation can be carried out by any technique known per se, such as filtration, and optionally washing of the precipitate with an appropriate solvent, in which the compound of formula (IA) is not soluble. The solvent used for the salification step can thus in particular be used.

Thus, according to another of its subjects, the present invention also relates to the compound of formula (IA):

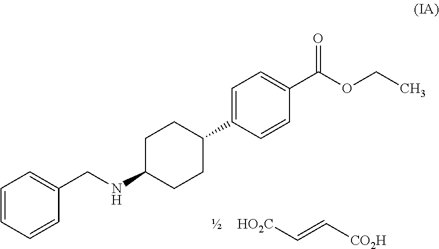

This compound is of use as an intermediate in the preparation of the compound of formula (A), by application or adaptation of the methods described in WO 02/44139, WO 03/099819 and WO 03/099772.

According to another subject, the present application also relates to a method for preparing the compound of formula (A) or a pharmaceutically acceptable salt thereof, comprising the preparation of the compound of formula (I) from the compound of formula (IA) as described above.

Preferably, the method for preparing the compound of formula (A), or a pharmaceutically acceptable salt thereof, also comprises the step of preparing the compound of formula (IA) as described above.

The method for preparing the compound of formula (A) also comprises the following steps:
coupling of the compound of formula (I) thus obtained from the compound of formula (IA), with the compound of formula (IV):

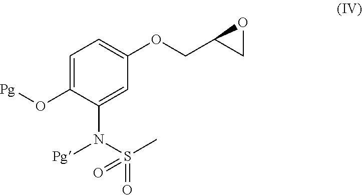

in which:
Pg represents an alcohol-function-protecting group;
Pg′ represents a hydrogen atom or an amine-function-protecting group;
deprotection of the protective groups present in the compound obtained so as to obtain the compound of formula (A); then, optionally,
salification, more particularly with hydrochloric acid, so as to obtain the hydrochloride of the compound of formula (A) desired.

Preferably, Pg represents benzyl; preferably, Pg′ represents H or a —C(═O) (OR) group where R represents a (C₁-C₄) alkyl group, more particularly tert-butyl.

The method for preparing the compound of formula (A) is represented in the scheme below:

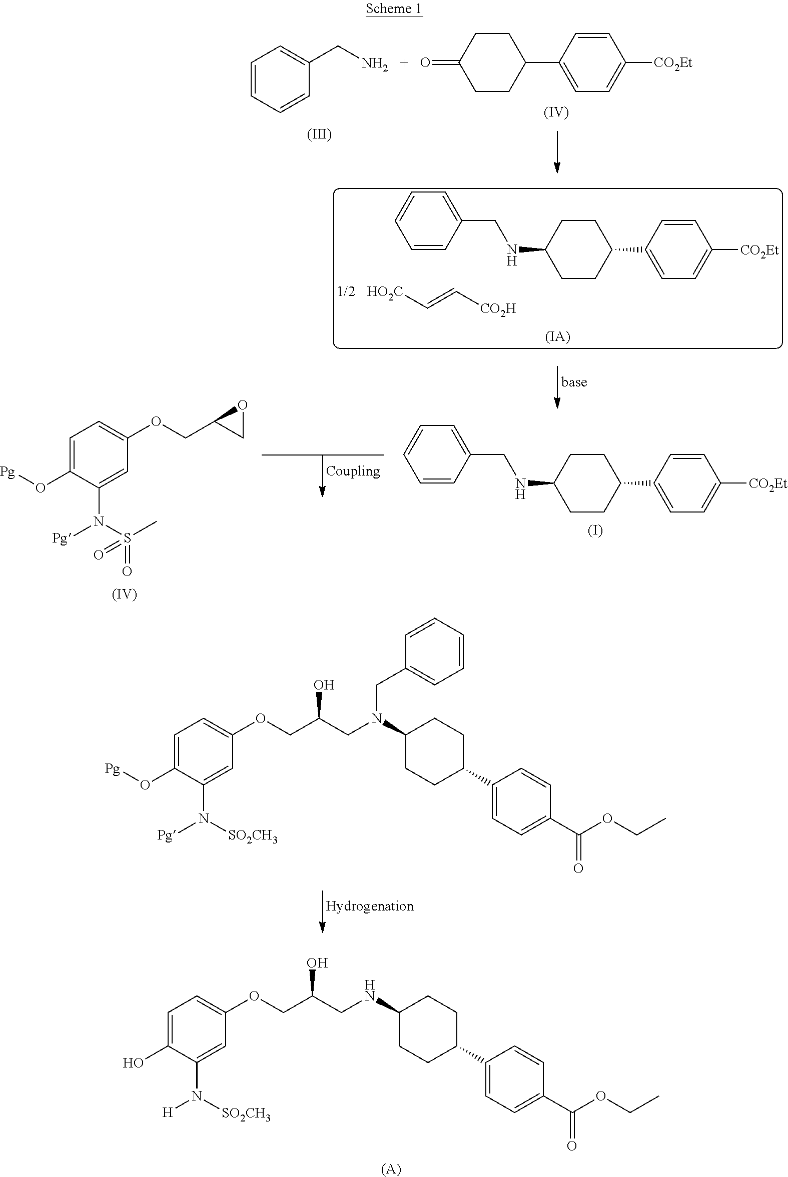

The present invention also relates to the use of the compound of formula (IA) for preparing the compound of formula (A) or a pharmaceutically acceptable salt thereof.

In the foregoing, the term “protective group” is intended to mean a group which makes it possible, firstly, to protect a reactive function, such as a hydroxyl or an amine, during a synthesis and, secondly, to regenerate the intact reactive function at the end of synthesis. Examples of protective groups and also of the methods of protection and deprotection are given in Protective Groups in Organic Synthesis, Green et al., 2nd Edition (John Wiley & Sons, Inc., New York).

The following examples illustrate the invention without, however, limiting it.

In these examples and in the description, the following abbreviations are used:

DCM: dichloromethane.

The mass spectra are measured in “Electrospray” (ES) ionization mode.

The proton nuclear magnetic resonance (¹H NMR) spectra are recorded at 200 MHz or at 300 MHz in DMSO-d₆ or in CDCl₃. The chemical shifts δ are expressed in parts per million (ppm).

The signals observed in NMR are expressed in the following way: s: singlet; bs: broad singlet; d: doublet; d.d.: double of doublet; t: triplet; dt: double of triplet; q: quadruplet; up: unresolved peak; mt: multiplet.

*=nonintegratable owing to interference with a broad peak due to water.

The mass spectra were obtained using a Thermoelectron LCQ DecaXP MCX ion trap spectrometer equipped with an ESI source.

The HPLC was carried out by means of a Thermoelectron Surveyor system equipped with a diode array detector.

The thin layer chromatography was carried out on Merck Silica gel 60 silica gel TLC plates. The silica gel for the flash column chromatography is sold by Biotage.

All the solvents used are of “reagent grade” or “HPLC grade” purity.

### EXAMPLE 1

### Step 1: Synthesis of the Compound of Formula (IA)

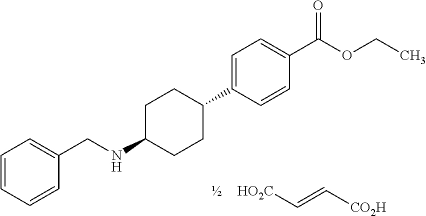

In a reactor, 3 g (12.18 mmol.) of 4-(4-ethoxycarbonyl-phenyl)cyclohexanone are introduced into 36 ml of trimethyl orthoformate, followed by 1.56 g (14.61 mmol., 1.2 eq.) of N-benzylamine.

The reaction medium is heated, under nitrogen, for 12 hours and then returned to ambient temperature and subsequently evaporated in a rotary evaporator until an oil is obtained. A residue is used as is in the next step.

The imine formed is taken up in 50 ml of ethanol. The resulting mixture is cooled to 5° C. and 0.460 g (12.18 mmol., 1 eq.) of sodium borohydride is added, under nitrogen, for 15 minutes. The reaction medium is left stirring for 2 hours at 5° C. 15 ml of water are then added and the resulting mixture is left stirring for 15 minutes and then extracted with ethyl acetate (50 ml). The organic phase is washed with 15 ml of water and then with 15 ml of a saturated sodium chloride solution. The resulting product is dried over magnesium sulfate, filtered, and then evaporated in a rotary evaporator. The oily residue (trans/cis mixture of the two amines, in an 85/15 ratio) is used as is in the salification step. The reaction crude is taken up in 50 ml of ethanol. The resulting mixture is heated to 50° C. 0.710 g (0.5 eq.) of fumaric acid is added. The medium becomes soluble, and is left to return to ambient temperature. The precipitate obtained is cooled to 5° C. Filtration is carried out and the crystals are washed with twice 5 ml of ice-cold ethanol. Drying is carried out under vacuum at 50° C. 2.40 g of the hemifumarate salt of 4-[trans-4-[(phenylmethyl)amino]-cyclohexyl]benzoic acid ethyl ester are obtained.

### Step 2: Synthesis of the 4-[trans-4-[(phenylmethyl)-amino]cyclohexyl]benzoic acid ethyl ester compound of formula (1)

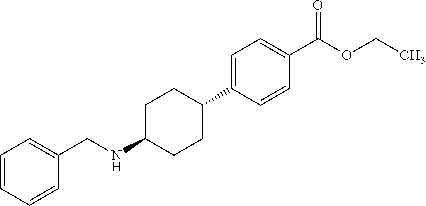

In a 100 ml three-necked flask, 2 g (5.06 mmol.) of the hemifumarate salt of 4-[trans-4-[(phenylmethyl)amino]-cyclohexyl]benzoic acid ethyl ester are introduced into 50 ml of dichloromethane. 10 ml of a 28% aqueous ammonia solution are then added and the mixture is left stirring at ambient temperature for 30 minutes. The two phases are left to separate by settling out, and then the organic phase is washed with water and subsequently dried with magnesium sulfate. Filtration is carried out and the solution is evaporated in a rotary evaporator. 1.60 g of 4-[trans-4-[(phenylmethyl)amino]cyclohexyl]-benzoic acid ethyl ester are obtained in the form of a white solid, i.e. a yield of 95%. Melting point=74-76° C., T.L.C.: (95:5:0.5 CH₂Cl₂/MeOH/NH₃) R_{f} trans=0.40.

The compound thus obtained can be used to prepare the compound of formula (A) by application or adaptation of the methods described in WO 02/44139, WO 03/099772 and WO 03/099819, or according to the following steps 3 and 4.

### Step 3: Synthesis of 4-[trans-4-[(phenylmethyl)-(2S)-3-(4-benzyloxy-3-methanesulfonylaminophenoxy)-2-hydroxypropyl]amino]cyclohexyl)benzoic acid ethyl ester

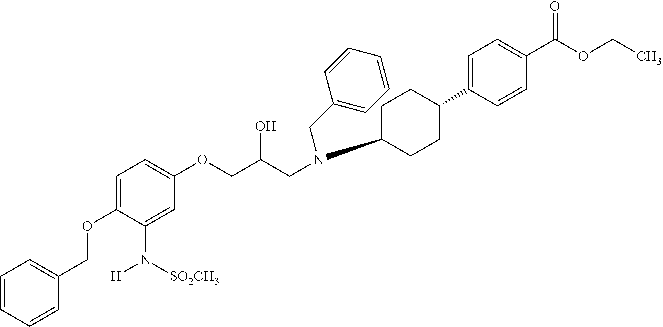

A mixture of N-[2-benzyloxy-5-((S)-1-oxiranylmethoxy)-phenyl]methanesulfonamide (9.4 kg), of 4-[trans-4-[(phenymethyl)amino]cyclohexyl]benzoic acid (7.05 kg) and of Li-triflate (lithium trifluoromethanesulfonate) (330 g) in a solution of toluene (90 l) is refluxed for 8 h. It is allowed to return to ambient temperature, and then the mixture is diluted in toluene (60 l) and extracted with water (15 l). The organic phase is concentrated under vacuum to a volume of 55 l. Crystallization of the product begins when the concentrate is returned to ambient temperature. The crystallized mixture is stirred at 5° C. for 90 minutes, and the product is filtered, washed with toluene (6 l) and dried under vacuum at 50° C. Yield: 12.84 kg, colorless crystals, melting point: 99-100° C.

### Step 4: Synthesis of the hydrochloride of the compound of formula (A) (4-[trans-4-[(S)-2-hydroxy-3-(4-hydroxy-3-methanesulfonylaminophenoxy)propylamino]cyclohexyl]-benzoic acid ethyl ester hydrochloride)

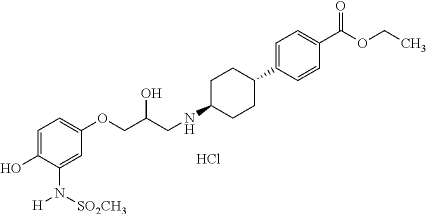

A mixture of trans-4-(4-{phenylmethyl-[(2S)-3-(4-benzyloxy-3-methanesulfonylaminophenoxy)-2-hydroxy-propyl]amino}cyclohexyl)benzoic acid ethyl ester (10 kg), of Pd(OH)₂ 20% on carbon (Degussa E101 NE/W, 0.52 kg) and of acetic acid (48 kg) is hydrogenated in an autoclave at 50° C./8 bar for 2 h and then the mixture is allowed to return to ambient temperature overnight under pressure. The mixture is filtered twice and the equipment is rinsed with acetic acid (25 kg). The filtrates are concentrated virtually to dryness, under vacuum, and then ethanol (45 kg) is added, followed by 30% HCl (2 kg). After the addition of crystal seeds, the mixture is stirred at 45° C. for 3 h and at 0° C. for 14 h. The crystals are filtered off, washed with ethanol (12 kg) and dried under vacuum at 40° C. Yield: 7.58 kg, colorless crystals, melting point: 192-193° C.

1. A method for preparing the compound of formula (IA): 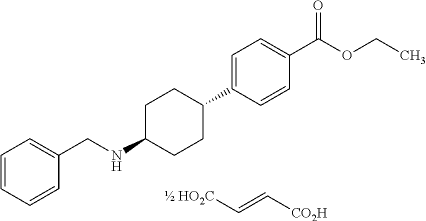
comprising the reaction of a compound of formula (II) with a compound of formula (III) 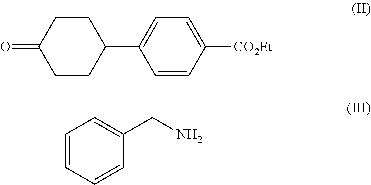
characterized in that:
said reaction is followed by a step of salification by addition of fumaric acid.

2. The method as claimed in claim 1, such that the reaction of the compound of formula (II) and of the compound of formula (III) is carried out in the presence of NaBH₄ or NaBH₃CN.

3. The method as claimed in claim 1, such that the reaction of the compound of formula (II) and of the compound of formula (III) is carried out in ethanol and/or trimethyl orthoformate.

4. The method as claimed in any one of the preceding claims, such that the salification is carried out by addition of half an equivalent of fumaric acid, relative to the compound of formula (II).

5. The method as claimed in claim 1, 2 or 3, such that the reaction of the compound of formula (II) and of the compound of formula (III) results in the formation of the compound of formula (I): 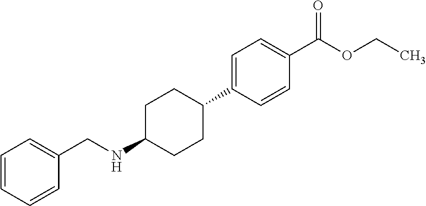
in cis/trans mixture form.

6. The method as claimed in any one of the preceding claims, such that it comprises the step of separating the compound of formula (IA) at the end of the salification.

7. The method as claimed in claim 6, such that the separation is carried out by filtration.

8. A method for preparing the compound of formula (I): 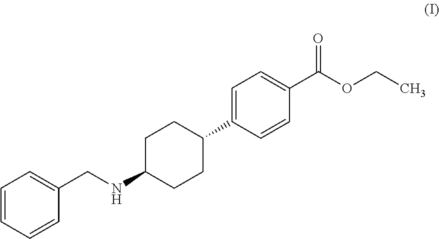
from the compound of formula (IA): 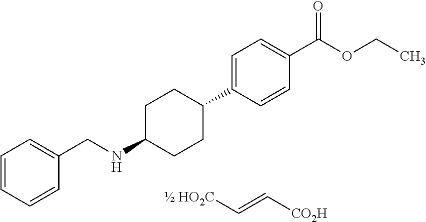
by addition of a base.

9. The method as claimed in claim 8, such that said base is aqueous ammonia or NH₄OH.

10. The method as claimed in any one of the preceding claims, such that it comprises isolating the compound obtained.

11. A compound of general formula (IA):

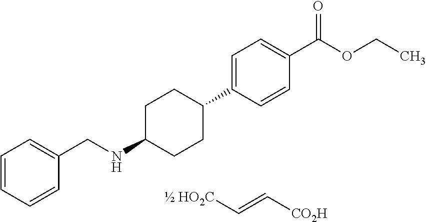

12. A method for preparing a compound of formula (A): 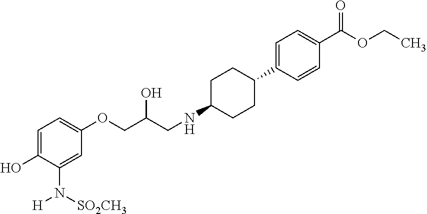
or a pharmaceutically acceptable salt thereof, comprising the preparation of the compound of formula (I): 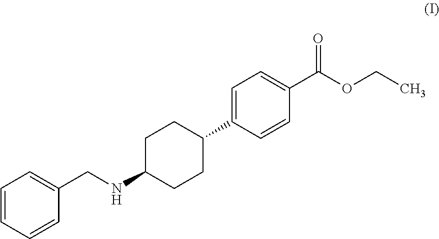
from the compound of formula (IA): 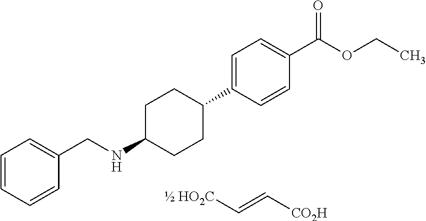
as claimed in claim 8 or 9.

13. The method for preparing a compound of formula (A) as claimed in claim 12, comprising the preparation of the compound of formula (IA) as claimed in any one of claims 1 to 7.

14. The method for preparing a compound of formula (A) as claimed in claim 12 or 13, comprising the subsequent steps of:
coupling of the compound of formula (I) and of the compound of formula (IV): 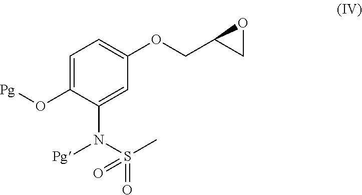
in which:
Pg represents an alcohol-function-protecting group;
Pg′ represents a hydrogen atom or an amine-function-protecting group;
deprotection of the protective groups present in the compound obtained so as to obtain the compound of formula (A); then, optionally,
salification, so as to obtain the salt of the compound of formula (A) desired.

15. The method as claimed in claim 14, such that Pg represents a benzyl group.

16. The method as claimed in claim 14 or 15, such that Pg′ represents H or a —C(═O) (OR) group where R represents a (C₁-C₄) alkyl group.

17. The use of the compound of formula (IA): 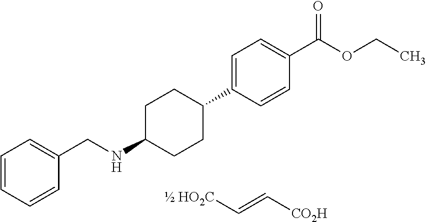
for preparing a compound of formula (A): 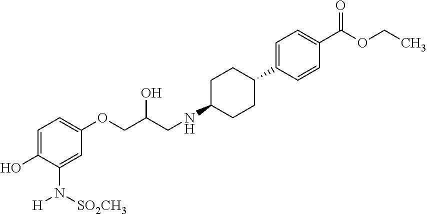
or a pharmaceutically acceptable salt thereof.
